(19) United States
(12) Patent Application Publication
(10) Pub. No.: US 2025/0268456 A1
(43) Pub. Date: Aug. 28, 2025

(54) TECHNIQUES FOR NASAL ENDOSCOPY PROCEDURE GUIDANCE AND DIAGNOSTICS

(71) Applicant: Scopi Inc., Loomis, CA (US)

(72) Inventors: Anna Sophie von Wendorff, Loomis, CA (US); Michael Brent Wilson, Salt Lake City, UT (US); Abdeslem Smahi, Lille (FR)

(21) Appl. No.: 19/060,325

(22) Filed: Feb. 21, 2025

Related U.S. Application Data
(60) Provisional application No. 63/557,221, filed on Feb. 23, 2024.

(51) Int. Cl. A61B 1/00; A61B 1/233

(52) U.S. Cl. CPC A61B 1/00055; A61B 1/00006; A61B 1/000094; A61B 1/000096; A61B 1/0004; A61B 1/0005; A61B 1/233

(57) ABSTRACT

Methods, systems, and devices for nasal endoscopy procedures and diagnostics are described. A system may acquire a set of images of a nasal cavity of a user using a nasal endoscopy device. The system may input the set of images into one or more machine learning models, the machine learning models trained using a set of tags associated with previous nasal endoscopy procedures. The system may determine an anatomical location of the nasal endoscopy device within the nasal cavity of the user using the machine learning models. The system may then display endoscope feedback associated with the set of images, wherein the endoscope feedback includes the anatomical location of the nasal endoscopy device, an instruction for moving the nasal endoscopy device, an anatomical structure depicted within the set of images, a clinical guidance or diagnosis of a medical condition depicted within the set of images, or any combination thereof.

### CROSS REFERENCE

The present Application for Patent claims priority to U.S. Provisional Patent Application No. 63/557,221 by von Wendorff et al., entitled “TECHNIQUES FOR NASAL ENDOSCOPY PROCEDURE GUIDANCE AND DIAGNOSTICS,” filed Feb. 23, 2024, which is assigned to the assignee hereof and expressly incorporated by reference herein.

### FIELD OF TECHNOLOGY

The following relates to nasal endoscopy procedures, including techniques for nasal endoscopy procedure guidance and diagnostics.

### BACKGROUND

Endoscopes are minimally invasive medical devices used to examine the internal anatomy of a patient. However, nasal endoscopy devices are typically only used by trained specialists. As such, primary care physicians typically have to refer their patients to specialists in order for the patients to undergo nasal endoscopy procedures. This increases the cost of nasal endoscopy procedures, and delays medical diagnoses for the patient.

### BRIEF DESCRIPTION OF THE DRAWINGS

FIG. 1 shows an example of a system that supports techniques for nasal endoscopy procedure guidance and diagnostics in accordance with aspects of the present disclosure.

FIG. 2 shows an example of a flow diagram that supports techniques for nasal endoscopy procedure guidance and diagnostics in accordance with aspects of the present disclosure.

FIG. 3 shows a block diagram of an apparatus that supports techniques for nasal endoscopy procedure guidance and diagnostics in accordance with aspects of the present disclosure.

FIG. 4 shows a block diagram of a nasal endoscopy application that supports techniques for nasal endoscopy procedure guidance and diagnostics in accordance with aspects of the present disclosure.

FIG. 5 shows a diagram of a system including a device that supports techniques for nasal endoscopy procedure guidance and diagnostics in accordance with aspects of the present disclosure.

FIGS. 6 through 8 show flowcharts illustrating methods that support techniques for nasal endoscopy procedure guidance and diagnostics in accordance with aspects of the present disclosure.

### DETAILED DESCRIPTION

Endoscopes are minimally invasive medical devices used to examine the internal anatomy of a patient. For example, nasal endoscopy devices may be used to collect video/images of a nasal canal of a patient during a nasal endoscopy procedure. However, nasal endoscopy devices are typically only used by trained nasal specialists (e.g., ear, nose, and throat (ENT) specialists). As such, primary care physicians typically have to refer their patients to nasal specialists in order for the patients to undergo nasal endoscopy procedures. This increases the cost of nasal endoscopy procedures, and delays medical diagnoses for the patient. Moreover, current nasal endoscopy devices are extremely expensive and bulky. As such, there is a need to reduce the size and cost of nasal endoscopy devices. Further, there is a need for improved methods and techniques that empower primary care physicians to perform nasal endoscopy procedures. In other words, there is a desire to make nasal endoscopy devices more accessible and usable to primary care physicians so that primary care physicians (and not just specialists) may perform nasal endoscopy procedures.

Accordingly, aspects of the present disclosure are directed to techniques for performing nasal endoscopy procedures using artificial intelligence (AI). In particular, aspects of the present disclosure are directed to techniques for leveraging machine learning models and other forms of AI to analyze images collected during nasal endoscopy procedures, and to provide feedback to an operator of the nasal endoscopy procedure (and/or other clinician analyzing images collected during the nasal endoscopy procedure). In some cases, techniques described herein may be used to generate real- time (and/or near-real time) feedback during a nasal endoscopy procedure to instruct the operator on how to effectively perform the nasal endoscopy procedure, understand and analyze the images collected during the nasal endoscopy procedure, or both. That is, aspects of the present disclosure may be used to provide real time and/or near-real time feedback during the course of a nasal endoscopy procedure to help the clinician perform the nasal endoscopy procedure in a safe and efficient manner.

For example, using machine learning models, techniques described herein may overlay video collected via a nasal endoscope with operator feedback that indicates the current anatomical location of the nasal endoscope, and indicates anatomical structures or medical conditions depicted within the video. In such cases, the operator feedback may instruct the operator of the nasal endoscopy device on how to move/manipulate the nasal endoscopy device in an efficient and safe manner that will enable the operator (or another clinician) to effectively evaluate the images and feedback to perform medical diagnoses and suggest appropriate medical treatments.

Aspects of the disclosure are initially described in the context of systems supporting nasal endoscopy procedures. Additional aspects of the present disclosure are described in the context of an example process flow. Aspects of the disclosure are further illustrated by and described with reference to apparatus diagrams, system diagrams, and flowcharts that relate to techniques for nasal endoscopy procedure guidance and diagnostics.

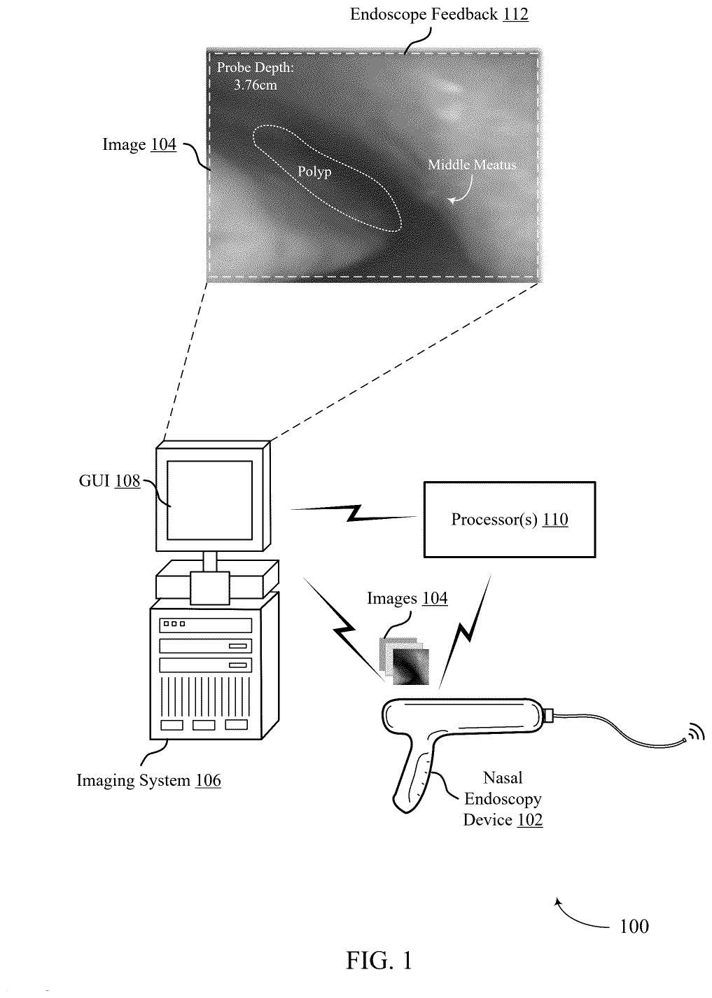

FIG. 1 shows an example of a system 100 that supports techniques for nasal endoscopy procedure guidance and diagnostics in accordance with aspects of the present disclosure. The system 100 may include an example of a system that is used to perform nasal endoscopy procedures and diagnostics in accordance with aspects of the present disclosure.

The system 100 may include various components that are configured to perform nasal endoscopy procedures and diagnostics, such as a nasal endoscopy device 102, an imaging system 106, and one or more processors 110. In some aspects, the nasal endoscopy device 102 may include an otoscope, a nasoendoscope, a rhinoscope, a laryngoscope, or any combination thereof.

The respective components may be communicatively coupled with one another via wireless and/or wired connections. In some cases, the one or more processors 110 may be associated with a dedicated processing unit or remote server (e.g., cloud-based server). In other cases, the one or more processors 110 maybe associated with or included within the nasal endoscopy device 102, the imaging system 106, or both. As such, while the one or more processors 110 are shown as being separate from the nasal endoscopy device 102 and the imaging system 106, this is solely for illustrative purposes.

The system 100 may acquire a set of images 104 of a nasal cavity of a user during a nasal endoscopy procedure using a nasal endoscopy device 102, and may transmit, to a graphical user interface (GUI) 108 of the imaging system 106, a first instruction configured to cause the GUI 108 to display the set of images 104 to an operator of the nasal endoscopy device 102, a clinician associated with the nasal endoscopy device 102, or both. The system 100 may input the set of images 104 into one or more machine learning models (e.g., during performance of the nasal endoscopy procedure). The machine learning models may be trained using a set of tags indicating anatomical structures, medical conditions, or both, depicted within a set of reference images associated with nasal cavities of a set of users. The system 100 may determine, using the one or more machine learning models, an anatomical location of the nasal endoscopy device 102 within the nasal cavity of the user based on inputting the set of images 104 into the one or more machine learning models.

For the purposes of the present disclosure, the term “reference images” may refer to images of nasal cavities (and/or other anatomical locations) that have been labeled/tagged with anatomical locations, anatomical structures, and/or diagnoses/medical conditions corresponding with the respective images. In some cases, aspects of the present disclosure that are implemented in the context of nasal endoscopy may use machine learning models that are trained using images of other anatomical locations of other users. In particular, it has been found that cross-training between different anatomies may increase accuracy of medical diagnoses. For instance, a model that is first trained to detect cervical cancer may better identify polyps in a nasal cavity as compared to a model that was originally trained on images of nasal cavities.

The system 100 may then transmit, to the GUI 108 of the imaging system 106 and based on the determined anatomical location, a second instruction configured to cause to GUI 108 to display endoscope feedback 112 associated with the set of images 104. The endoscope feedback 112 may include an indication of the anatomical location of the nasal endoscopy device 102, an instruction for moving the nasal endoscopy device 102 relative to the anatomical location, an indication of an anatomical structure depicted within the set of images 104, a diagnosis and/or clinical guidance of a medical condition within the nasal cavity depicted within the set of images 104, or any combination thereof. For the purposes of the present disclosure, the term “clinical guidance” may refer to information that indicates a relative severity or likelihood of a particular medical condition or diagnosis that may help a clinician perform the nasal endoscopy procedure and/or identify/treat a particular medical condition. For instance, the endoscope feedback 112 may provide clinical guidance that indicates “Degree of nasal congestion: mild,” or “possible indication of cancer.” In some aspects, the anatomical structure may include a structural feature of the nasal cavity, a polyp, mucus, or any combination thereof.

In some aspects, the endoscope feedback 112 may be overlaid on top of the set of images 104 in real time or near-real time during the nasal endoscopy procedure. In some implementations, the endoscope feedback 112 may additionally or alternatively be based on other information (e.g., physiological information) or data sources, such as microphones, oxygen saturation oximeters, blood pressure monitors, heart rate monitors, perfusion monitors, tissue oxygenation spectroscopy devices, rhinomanometry, pH or impedance monitoring, or any combination thereof. For instance, an SpO2 monitoring device may monitor the blood oxygen saturation level of the user throughout the nasal endoscopy procedure, where the endoscope feedback 112 may may be based on the collected blood oxygen saturation level of the user. For instance, the endoscope feedback 112 may instruct the clinician/operator to terminate the nasal endoscopy procedure if the user's blood oxygenation saturation levels fall below some threshold level. In other cases, the endoscope feedback 112 may be provided after the nasal endoscopy procedure in order to enable a clinician to perform medical diagnoses for the patient and/or to evaluate the operator who performed the nasal endoscopy procedure.

In some aspects, the one or more processors 110 of the system 100 may transmit a signal to the nasal endoscopy device 102 to cause the nasal endoscopy device 102 to modify one or more operational parameters of the nasal endoscopy device 102 based on the anatomical structure depicted within the set of images 104, the clinical guidance/diagnosis of the medical condition within the nasal cavity depicted within the set of images 104, or both. That is, the system 100 may modify the operational parameters of the nasal endoscopy device 102 in order to improve a quality of the images 104 collected by the device for imaging particular anatomical structures, performing diagnoses, etc. In some aspects, the operational parameters of the nasal endoscopy device 102 may include a wavelength of light used by one or more light-emitting components of the nasal endoscopy device 102, a brightness of the one or more light-emitting components, a frame rate for collecting images 104, a color saturation setting, a level of suction (e.g., level of suction provided by the nasal endoscopy device 102), an irrigation setting of the nasal endoscopy device 102, a medication delivery setting for delivering medication by the nasal endoscopy device 102, one or more imaging settings of one or more light-receiving components of the nasal endoscopy device 102, or any combination thereof. In additional or alternative implementations, the nasal endoscopy device 102 may automatically capture and/or send an image/video for analysis.

In some aspects, the one or more processors 110 of the system 100 may determine, using the one or more machine learning models, a first set of characteristics associated with the anatomical structure depicted within the set of images 104. The processors 110 may acquire additional images 104 of the anatomical structure collected via the nasal endoscopy device 102 using the one or more modified operational parameters and based on transmitting the signal to the nasal endoscopy device 102, and may input the additional images 104 into the one or more machine learning models. In this example, the processors 110 may determine, using the one or more machine learning models, a second set of characteristics associated with the anatomical structure depicted within the additional images 104. In other words, the system 100 may modify the operational parameters of the nasal endoscopy device 102 in order to more effectively/efficiently evaluate anatomical structures (e.g., polyps) depicted within the images 104.

In some aspects, the one or more processors 110 of the system 100 may acquire additional images 104 of the nasal cavity of the user collected via the nasal endoscopy device 102 using the one or more modified operational parameters of the nasal endoscopy device 102. The processors 110 may input the additional images 104 into the one or more machine learning models, and determine, using the one or more machine learning models, an adjusted clinical guidance/diagnosis of the medical condition based on inputting the additional images 104 into the one or more machine learning models. That is, by adjusting the operational parameters of the nasal endoscopy device 102, the system 100 may be able to provide updated (e.g., improved, modified) clinical guidance/medical diagnoses.

In some aspects, the one or more processors 110 of the system 100 may acquire additional images 104 of the nasal cavity of the user collected via the nasal endoscopy device 102 using the one or more modified operational parameters and based on transmitting the signal to the nasal endoscopy device 102. The processors 110 may input the additional images 104 into the one or more machine learning models. The system 100 may transmit, to the GUI 108 of the imaging system 106 and based on inputting the additional images 104 into the one or more machine learning models, a third instruction configured to cause to GUI 108 to display additional endoscope feedback 112 associated with the additional images 104.

In some aspects, the one or more processors 110 of the system 100 may identify, using the one or more machine learning models, an image 104 of the set of images 104 that depicts a reference structure within the nasal cavity of the user. The processors 110 may generate an association between the image 104 and a reference anatomical location, and determine the anatomical location of the nasal endoscopy device 102 within the nasal cavity based on the reference anatomical location and a comparison between the image 104 and the set of images 104. In other words, throughout the course of the nasal endoscopy procedure, the system 100 may determine the location of the nasal endoscopy device 102 within the nasal cavity by comparing images 104 collected by the nasal endoscopy device 102 with the location of some “reference anatomical location” depicted in one (or more) of the images captured during the nasal endoscopy procedure. In some aspects, the reference structure may include a middle meatus of the user.

In some aspects, the one or more processors 110 of the system 100 may receive speed data, acceleration data, or both, associated with a movement of the nasal endoscopy device 102 within the nasal cavity of the user. The processors 110 may input the speed data, the acceleration data, or both, into the one or more machine learning models, where the anatomical location of the nasal endoscopy device 102 throughout the nasal endoscopy procedure may be determined, tracked, or otherwise monitored based on the reference anatomical location and the speed data, the acceleration data, or both. The processors 110 may generate, using the one or more machine learning models, a three-dimensional (3D) model of the nasal cavity of the user based on the set of images 104 and the speed data, the acceleration data, or both, where the anatomical location of the nasal endoscopy device 102 may be determined based on the 3D model. Further, the endoscope feedback 112 displayed to the operator of the nasal endoscopy device 102 may be based on the location of the nasal endoscopy device 102 within the nasal cavity and/or based on the location of the nasal endoscopy device 102 relative to some reference anatomical location/structure.

In some aspects, the one or more processors 110 of the system 100 may identify a set of anatomical structures or anatomical locations of the nasal cavity of the user depicted within the set of images 104, and may compare the set of anatomical structures or anatomical locations of the nasal cavity of the user with a set of reference structures or reference locations (e.g., middle meatus), where the endoscope feedback 112 may be based on the comparison. Stated differently, the system 100 may analyze the images 104 collected during the nasal endoscopy procedure to determine whether important or features and/or locations of the nasal cavity have been adequately imaged, and to ensure that the nasal endoscopy procedure collects sufficient data for performing clinical guidance/diagnoses.

In some aspects, the endoscope feedback 112 may include an indication of a completion of the nasal endoscopy procedure based on the set of anatomical structures or anatomical locations matching the set of reference structures or reference locations. For instance, if the system 100 determines that the images 104 collected by the nasal endoscopy device 102 have adequately captured all the “important” or “expected” locations/anatomical structures of the nasal cavity, the system 100 may provide endoscope feedback 112 indicating that the nasal endoscopy procedure has been performed successfully, and may be concluded. In other cases, the endoscope feedback 112 may include directions for the operator of the nasal endoscopy device 102 move the nasal endoscopy device 102 to a different anatomical location based on the set of anatomical structures or anatomical locations failing to match the set of reference structures or reference locations. That is, if the system 100 determines that there are certain locations and/or anatomical structures that have not been adequately captured via the collected images 104, the endoscope feedback 112 may indicate for the operator to move the nasal endoscopy device 102 to a particular location (and/or adjust the orientation or operational parameters of the nasal endoscopy device 102) to capture images of the missing locations/anatomical structures.

In some aspects, the one or more processors 110 of the system 100 may determine, using the one or more machine learning models, a difference between a viewpoint of the anatomical structure of the user depicted within the set of images 104 and a reference viewpoint of one or more corresponding anatomical structures of the set of users depicted within the set of reference images. In such cases, the endoscope feedback 112 may include directions for the operator to manipulate the nasal endoscopy device 102 to match the viewpoint of the anatomical structure with the reference viewpoint. By assisting the operator of the nasal endoscopy device to match the viewpoint of the images with the reference viewpoint of the reference images, techniques described herein may enable the machine learning models to more effectively perform clinical guidance/diagnoses.

In some aspects, the endoscope feedback 112 may include directions for the operator to maintain the nasal endoscopy device 102 in a current anatomical location based on the anatomical structure depicted within the set of images 104. That is, the endoscope feedback 112 may instruct the operator to maintain the nasal endoscopy device 102 in a current anatomical location in order to capture additional images 104 of a particular anatomical location and/or anatomical structure.

In some aspects, the one or more processors 110 of the system 100 may determine, using the one or more machine learning models, a relative priority of the set of images 104 based on the anatomical location associated with each of the set of images 104, anatomical structures depicted within each of the set of images 104, or both. That is, the system 100 may prioritize the images 104 collected during the nasal endoscopy procedure based on a relative importance or significance of the anatomical locations/structures depicted in the respective images. For instance, the system 100 may prioritize, categorize, or otherwise label a subset of images 104 which are most indicative of a particular medical condition, or a subset of images 104 which most support a particular medical diagnoses.

In some aspects, the one or more processors 110 of the system 100 may acquire, during a reference nasal endoscopy procedure, a set of reference images of the nasal cavity of the user. In this example, the processors 110 may determine, using the one or more machine learning models and based on a comparison between the set of images 104 and the set of reference images, a change in one or more characteristics of the anatomical structure, a change in the clinical guidance/diagnosis of the medical condition, or both.

In some aspects, the one or more processors 110 of the system 100 may receive the set of reference images depicting the nasal cavities of the set of users, and tag the set of reference images with the set of tags based on the anatomical structures, medical conditions, or both, depicted in the respective images to generate a set of tagged reference images 104. The processors 110 may train the one or more machine learning models to identify, diagnose, or otherwise provide clinical guidance regarding medical conditions during nasal endoscopy procedures based on inputting the set of tagged reference images into the one or more machine learning models, wherein determining the anatomical location of the set of images 104 may be based on comparing, using the one or more machine learning models, the set of images 104 with the set of tagged reference images.

In some aspects, the one or more processors 110 of the system 100 may compare the set of images 104 with the set of reference images using the one or more machine learning models. The processors 110 may select, based on the comparison, at least one image from the set of reference images 104 that satisfies one or more similarity criteria with the set of images 104. In such cases, the processors 110 may transmit, to the GUI 108 of the imaging system 106, a third instruction configured to cause the GUI 108 to display the selected at least one image alongside the set of images 104.

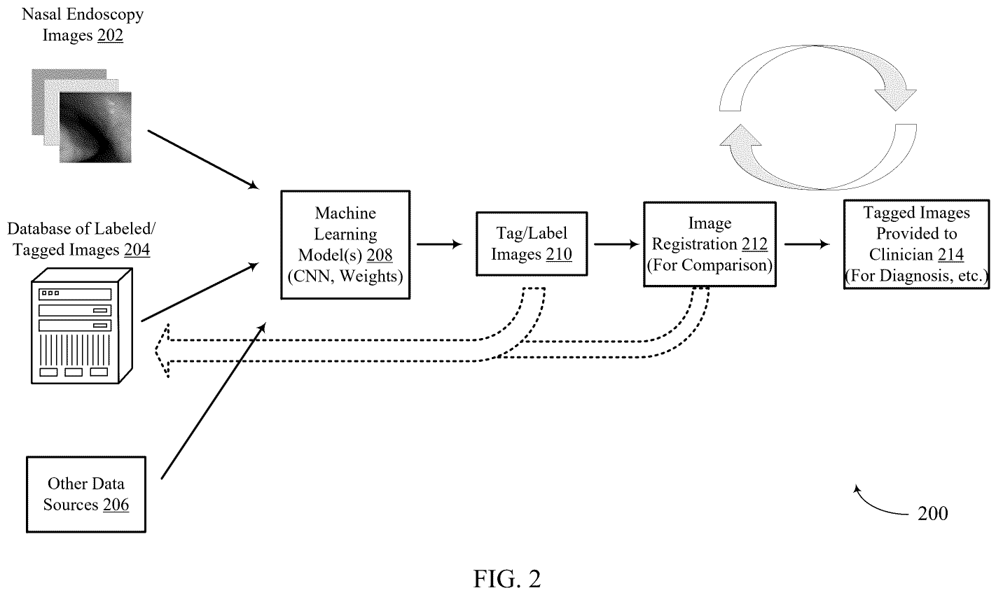

FIG. 2 shows an example of a process flow 200 that supports techniques for nasal endoscopy procedure guidance and diagnostics in accordance with aspects of the present disclosure. Aspects of the process flow 200 may implement, or be implemented by, aspects of the system 100.

The process flow 200 illustrates example steps/functions for performing nasal endoscopy procedures and diagnostics in accordance with aspects of the present disclosure. In particular, the process flow 200 illustrates steps/functions that may be carried out by the respective components of the system 100 shown and described in FIG. 2. For example, the nasal endoscopy images 202 shown and described in FIG. 2 may include examples of the images 104 shown and described in FIG. 1. Moreover, the machine learning models 208 shown and described in FIG. 2 may be examples of the machine learning models shown and described in FIG. 1.

In some aspects, nasal endoscopy images 202 (e.g., images 104 collected via a nasal endoscopy device 102) may be inputted into one or more machine learning models 208. In some aspects, labeled/tagged images 204 (e.g., reference images) may also be input into the machine learning models 208, as well as data/information from other data sources 206 (e.g., additional medical information or diagnoses, etc.). At 210, the nasal endoscopy images 202 may be tagged or labeled (e.g., tagging/labeling anatomical locations, anatomical structures, medical conditions, etc.). In some cases, the tagged/labeled images from step 210 may be provided back to the database and stored for future reference. At 212, the system may perform image registration (for comparison). At 214, the tagged images may be provided to a clinician (e.g., for diagnosis, medical treatment analysis, etc.).

In some aspects, steps of the process flow 200 may be performed in real-tome (and/or near-real time) during the performance of a nasal endoscopy procedure. That is, the nasal endoscopy images 202 may be collected during a nasal endoscopy procedure, input into machine learning models 208, and tagged/labeled in real time (and/or near-real time) during the course of the nasal endoscopy procedure. For example, in some cases, images 104 collected by a nasal endoscopy device 102 during a nasal endoscopy procedure may be tagged and/or labeled (step 210) my machine learning models in real time (and/or near-real time) during the course of the nasal endoscopy procedure. For instance, the system 100 may tag/label collected images 104, where the tags/labels may be displayed to the operator via the endoscopy feedback 112 (e.g., step 214).

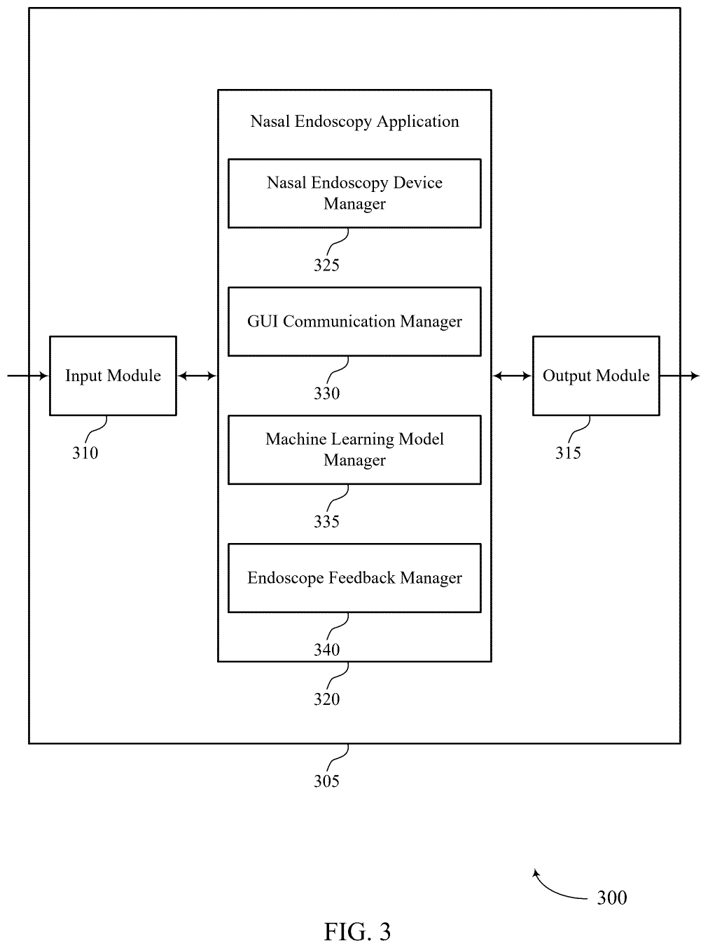

FIG. 3 shows a block diagram 300 of a device 305 that supports techniques for nasal endoscopy procedure guidance and diagnostics in accordance with aspects of the present disclosure. The device 305 may include an input module 310, an output module 315, and a nasal endoscopy application 320. The device 305, or one of more components of the device 305 (e.g., the input module 310, the output module 315, the nasal endoscopy application 320), may include at least one processor, which may be coupled with at least one memory, to support the described techniques. Each of these components may be in communication with one another (e.g., via one or more buses).

The input module 310 may provide a means for receiving information such as packets, user data, control information, or any combination thereof associated with various information channels (e.g., control channels, data channels, information channels related to illness detection techniques). Information may be passed on to other components of the device 305. The input module 310 may utilize a single antenna or a set of multiple antennas.

The output module 315 may provide a means for transmitting signals generated by other components of the device 305. For example, the output module 315 may transmit information such as packets, user data, control information, or any combination thereof associated with various information channels (e.g., control channels, data channels, information channels related to illness detection techniques). In some examples, the output module 315 may be co-located with the input module 310 in a transceiver module. The output module 315 may utilize a single antenna or a set of multiple antennas.

For example, the nasal endoscopy application 320 may include a nasal endoscopy device manager 325, a GUI communication manager 330, a machine learning model manager 335, an endoscope feedback manager 340, or any combination thereof. In some examples, the nasal endoscopy application 320, or various components thereof, may be configured to perform various operations (e.g., receiving, monitoring, transmitting) using or otherwise in cooperation with the input module 310, the output module 315, or both. For example, the nasal endoscopy application 320 may receive information from the input module 310, send information to the output module 315, or be integrated in combination with the input module 310, the output module 315, or both to receive information, transmit information, or perform various other operations as described herein.

The nasal endoscopy application 320 may support providing feedback associated with a nasal endoscopy procedure in accordance with examples as disclosed herein. The nasal endoscopy device manager 325 may be configured as or otherwise support a means for acquiring a plurality of images of a nasal cavity of a user using a nasal endoscopy device. The GUI communication manager 330 may be configured as or otherwise support a means for transmitting, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both. The machine learning model manager 335 may be configured as or otherwise support a means for inputting, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users. The machine learning model manager 335 may be configured as or otherwise support a means for determining, using the one or more machine learning models, an anatomical location of the nasal endoscopy device within the nasal cavity of the user based at least in part on inputting the plurality of images into the one or more machine learning models. The endoscope feedback manager 340 may be configured as or otherwise support a means for transmitting, to the GUI of the imaging system and based at least in part on the determined anatomical location, a second instruction configured to cause the GUI to display endoscope feedback associated with the plurality of images, wherein the endoscope feedback comprises an indication of the anatomical location of the nasal endoscopy device, an instruction for moving the nasal endoscopy device relative to the anatomical location, an indication of an anatomical structure depicted within the plurality of images, a diagnosis of a medical condition within the nasal cavity depicted within the plurality of images, or any combination thereof.

Additionally, or alternatively, the nasal endoscopy application 320 may support providing feedback associated with a nasal endoscopy procedure in accordance with examples as disclosed herein. The nasal endoscopy device manager 325 may be configured as or otherwise support a means for acquiring a plurality of images of a nasal cavity of a user using a nasal endoscopy device. The GUI communication manager 330 may be configured as or otherwise support a means for transmitting, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both. The machine learning model manager 335 may be configured as or otherwise support a means for inputting, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users. The endoscope feedback manager 340 may be configured as or otherwise support a means for generating endoscope feedback associated with the plurality of images based at least in part on inputting the plurality of images into the one or more machine learning models. The endoscope feedback manager 340 may be configured as or otherwise support a means for transmitting, to the GUI of the imaging system, a second instruction configured to cause the GUI to display the endoscope feedback associated with the plurality of images, wherein the endoscope feedback comprises an indication of an anatomical location of the nasal endoscopy device within the nasal cavity, an instruction for moving the nasal endoscopy device relative to the anatomical location, an indication of an anatomical structure depicted within the plurality of images, a diagnosis of a medical condition within the nasal cavity depicted within the plurality of images, or any combination thereof.

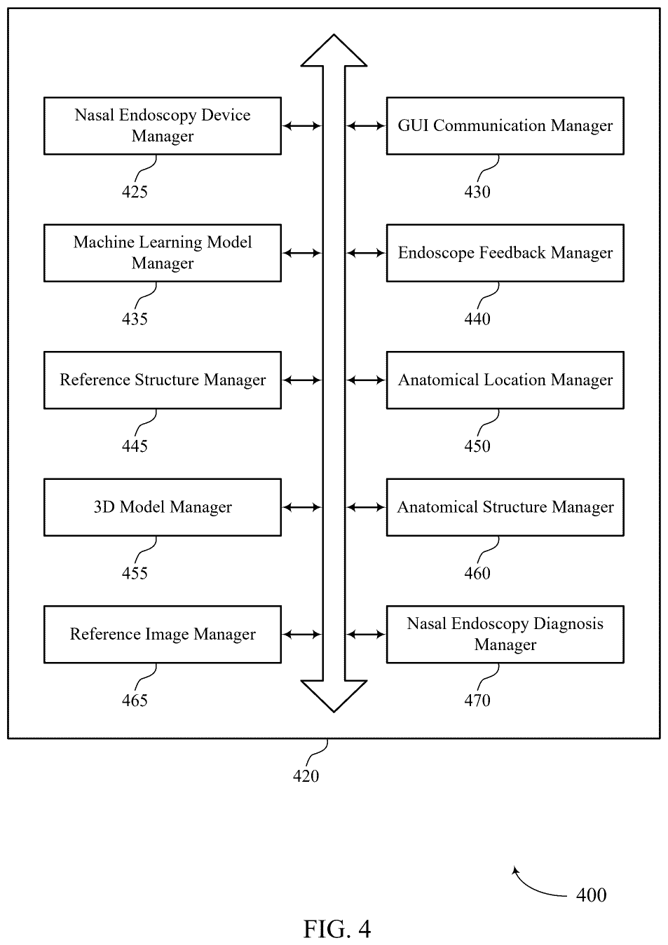

FIG. 4 shows a block diagram 400 of a nasal endoscopy application 420 that supports techniques for nasal endoscopy procedure guidance and diagnostics in accordance with aspects of the present disclosure. The nasal endoscopy application 420 may be an example of aspects of the nasal endoscopy application 320, as described herein. The nasal endoscopy application 420, or various components thereof, may be an example of means for performing various aspects of techniques for nasal endoscopy procedure guidance and diagnostics as described herein. For example, the nasal endoscopy application 420 may include a nasal endoscopy device manager 425, a GUI communication manager 430, a machine learning model manager 435, an endoscope feedback manager 440, a reference structure manager 445, an anatomical location manager 450, a 3D model manager 455, an anatomical structure manager 460, a reference image manager 465, a nasal endoscopy diagnosis manager 470, or any combination thereof. Each of these components, or components of subcomponents thereof (e.g., one or more processors, one or more memories), may communicate, directly or indirectly, with one another (e.g., via one or more buses).

The nasal endoscopy application 420 may support providing feedback associated with a nasal endoscopy procedure in accordance with examples as disclosed herein. The nasal endoscopy device manager 425 may be configured as or otherwise support a means for acquiring a plurality of images of a nasal cavity of a user using a nasal endoscopy device. The GUI communication manager 430 may be configured as or otherwise support a means for transmitting, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both. The machine learning model manager 435 may be configured as or otherwise support a means for inputting, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users. In some examples, the machine learning model manager 435 may be configured as or otherwise support a means for determining, using the one or more machine learning models, an anatomical location of the nasal endoscopy device within the nasal cavity of the user based at least in part on inputting the plurality of images into the one or more machine learning models. The endoscope feedback manager 440 may be configured as or otherwise support a means for transmitting, to the GUI of the imaging system and based at least in part on the determined anatomical location, a second instruction configured to cause the GUI to display endoscope feedback associated with the plurality of images, wherein the endoscope feedback comprises an indication of the anatomical location of the nasal endoscopy device, an instruction for moving the nasal endoscopy device relative to the anatomical location, an indication of an anatomical structure depicted within the plurality of images, a diagnosis of a medical condition within the nasal cavity depicted within the plurality of images, or any combination thereof.

In some examples, the nasal endoscopy device manager 425 may be configured as or otherwise support a means for transmitting a signal to the nasal endoscopy device to cause the nasal endoscopy device to modify one or more operational parameters of the nasal endoscopy device based at least in part on the anatomical structure depicted within the plurality of images, the diagnosis of the medical condition within the nasal cavity depicted within the plurality of images, or both.

In some examples, the machine learning model manager 435 may be configured as or otherwise support a means for determining, using the one or more machine learning models, a first set of characteristics associated with the anatomical structure depicted within the plurality of images. In some examples, the nasal endoscopy device manager 425 may be configured as or otherwise support a means for acquiring additional images of the anatomical structure collected via the nasal endoscopy device using the one or more modified operational parameters and based at least in part on transmitting the signal to the nasal endoscopy device. In some examples, the machine learning model manager 435 may be configured as or otherwise support a means for inputting the additional images into the one or more machine learning models. In some examples, the anatomical structure manager 460 may be configured as or otherwise support a means for determining, using the one or more machine learning models, a second set of characteristics associated with the anatomical structure depicted within the additional images.

In some examples, the nasal endoscopy device manager 425 may be configured as or otherwise support a means for acquiring additional images of the nasal cavity of the user collected via the nasal endoscopy device using the one or more modified operational parameters and based at least in part on transmitting the signal to the nasal endoscopy device. In some examples, the machine learning model manager 435 may be configured as or otherwise support a means for inputting the additional images into the one or more machine learning models. In some examples, the nasal endoscopy diagnosis manager 470 may be configured as or otherwise support a means for determining, using the one or more machine learning models, an adjusted diagnosis of the medical condition based at least in part on inputting the additional images into the one or more machine learning models.

In some examples, the nasal endoscopy device manager 425 may be configured as or otherwise support a means for acquiring additional images of the nasal cavity of the user collected via the nasal endoscopy device using the one or more modified operational parameters and based at least in part on transmitting the signal to the nasal endoscopy device. In some examples, the machine learning model manager 435 may be configured as or otherwise support a means for inputting the additional images into the one or more machine learning models. In some examples, the GUI communication manager 430 may be configured as or otherwise support a means for transmitting, to the GUI of the imaging system and based at least in part on inputting the additional images into the one or more machine learning models, a third instruction configured to cause the GUI to display additional endoscope feedback associated with the additional images.

In some examples, the one or more operational parameters of the nasal endoscopy device comprise a wavelength of light used by one or more light-emitting components of the nasal endoscopy device, a brightness of the one or more light-emitting components, a frame rate for collecting images, a color saturation setting, one or more imaging settings of one or more light-receiving components of the nasal endoscopy device, or any combination thereof.

In some examples, the reference structure manager 445 may be configured as or otherwise support a means for identifying, using the one or more machine learning models, an image of the plurality of images that depicts a reference structure within the nasal cavity of the user. In some examples, the anatomical location manager 450 may be configured as or otherwise support a means for generating an association between the image and a reference anatomical location, wherein determining the anatomical location of the nasal endoscopy device within the nasal cavity is based at least in part on the reference anatomical location and a comparison between the image and the plurality of images.

In some examples, the reference structure comprises a middle meatus of the user.

In some examples, the anatomical location manager 450 may be configured as or otherwise support a means for receiving speed data, acceleration data, or both, associated with a movement of the nasal endoscopy device within the nasal cavity of the user. In some examples, the machine learning model manager 435 may be configured as or otherwise support a means for inputting the speed data, the acceleration data, or both, into the one or more machine learning models, wherein the anatomical location of the nasal endoscopy device is determined based at least in part on the reference anatomical location and the speed data, the acceleration data, or both.

In some examples, the anatomical location manager 450 may be configured as or otherwise support a means for receiving speed data, acceleration data, or both, associated with a movement of the nasal endoscopy device within the nasal cavity during the nasal endoscopy procedure. In some examples, the machine learning model manager 435 may be configured as or otherwise support a means for inputting the speed data, the acceleration data, or both, into the one or more machine learning models. In some examples, the 3D model manager 455 may be configured as or otherwise support a means for generating, using the one or more machine learning models, a three-dimensional model of the nasal cavity of the user based at least in part on the plurality of images and the speed data, the acceleration data, or both, wherein determining the anatomical location of the nasal endoscopy device is based at least in part on the three-dimensional model.

In some examples, the anatomical structure manager 460 may be configured as or otherwise support a means for identifying a plurality of anatomical structures or anatomical locations of the nasal cavity of the user depicted within the plurality of images. In some examples, the anatomical structure manager 460 may be configured as or otherwise support a means for comparing the plurality of anatomical structures or anatomical locations of the nasal cavity of the user with a plurality of reference structures or reference locations, wherein the endoscope feedback is based at least in part on the comparison.

In some examples, the endoscope feedback comprises an indication of a completion of the nasal endoscopy procedure based at least in part on the plurality of anatomical structures or anatomical locations matching the plurality of reference structures or reference locations. In some examples, the endoscope feedback comprises directions for the operator of the nasal endoscopy device move the nasal endoscopy device to a different anatomical location based at least in part on the plurality of anatomical structures or anatomical locations failing to match the plurality of reference structures or reference locations.

In some examples, the machine learning model manager 435 may be configured as or otherwise support a means for determining, using the one or more machine learning models, a difference between a viewpoint of the anatomical structure of the user depicted within the plurality of images and a reference viewpoint of one or more corresponding anatomical structures of the plurality of users depicted within the set of reference images, wherein the endoscope feedback comprises directions for the operator to manipulate the nasal endoscopy device to match the viewpoint of the anatomical structure with the reference viewpoint.

In some examples, the endoscope feedback comprises directions for the operator to maintain the nasal endoscopy device in a current anatomical location based at least in part on the anatomical structure depicted within the plurality of images.

In some examples, the machine learning model manager 435 may be configured as or otherwise support a means for determining, using the one or more machine learning models, a relative priority of the plurality of images based at least in part on the anatomical location associated with each of the plurality of images, anatomical structures depicted within each of the plurality of images, or both.

In some examples, the reference image manager 465 may be configured as or otherwise support a means for acquiring, during a reference nasal endoscopy procedure, a plurality of reference images of the nasal cavity of the user. In some examples, the anatomical structure manager 460 may be configured as or otherwise support a means for determining, using the one or more machine learning models and based on a comparison between the plurality of images and the plurality of reference images, a change in one or more characteristics of the anatomical structure, a change in the diagnosis of the medical condition, or both.

In some examples, the second instruction causes the GUI of the imaging system to overlay the endoscope feedback on top of the plurality of images in real time or near-real time during the nasal endoscopy procedure.

In some examples, the anatomical structure comprises a structural feature of the nasal cavity, a polyp, or both.

In some examples, the reference image manager 465 may be configured as or otherwise support a means for receiving the set of reference images depicting the nasal cavities of the plurality of users. In some examples, the reference image manager 465 may be configured as or otherwise support a means for tagging the set of reference images with the set of tags based on the anatomical structures, medical conditions, or both, depicted in the respective images to generate a set of tagged reference images. In some examples, the machine learning model manager 435 may be configured as or otherwise support a means for training the one or more machine learning models to diagnose medical conditions during nasal endoscopy procedures based at least in part on inputting the set of tagged reference images into the one or more machine learning models, wherein determining the anatomical location of the plurality of images is based at least in part on comparing, using the one or more machine learning models, the plurality of images with the set of tagged reference images.

In some examples, the nasal endoscopy device comprises an otoscope, a nasoendoscope, a rhinoscope, a laryngoscope, or any combination thereof.

In some examples, the reference image manager 465 may be configured as or otherwise support a means for comparing the plurality of images with the set of reference images using the one or more machine learning models. In some examples, the reference image manager 465 may be configured as or otherwise support a means for selecting, based at least in part on the comparison, at least one image from the set of reference images that satisfies one or more similarity criteria with the plurality of images. In some examples, the GUI communication manager 430 may be configured as or otherwise support a means for transmitting, to the GUI of the imaging system, a third instruction configured to cause the GUI to display the selected at least one image alongside the plurality of images.

Additionally, or alternatively, the nasal endoscopy application 420 may support providing feedback associated with a nasal endoscopy procedure in accordance with examples as disclosed herein. In some examples, the nasal endoscopy device manager 425 may be configured as or otherwise support a means for acquiring a plurality of images of a nasal cavity of a user using a nasal endoscopy device. In some examples, the GUI communication manager 430 may be configured as or otherwise support a means for transmitting, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both. In some examples, the machine learning model manager 435 may be configured as or otherwise support a means for inputting, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users. In some examples, the endoscope feedback manager 440 may be configured as or otherwise support a means for generating endoscope feedback associated with the plurality of images based at least in part on inputting the plurality of images into the one or more machine learning models. In some examples, the endoscope feedback manager 440 may be configured as or otherwise support a means for transmitting, to the GUI of the imaging system, a second instruction configured to cause the GUI to display the endoscope feedback associated with the plurality of images, wherein the endoscope feedback comprises an indication of an anatomical location of the nasal endoscopy device within the nasal cavity, an instruction for moving the nasal endoscopy device relative to the anatomical location, an indication of an anatomical structure depicted within the plurality of images, a diagnosis of a medical condition within the nasal cavity depicted within the plurality of images, or any combination thereof.

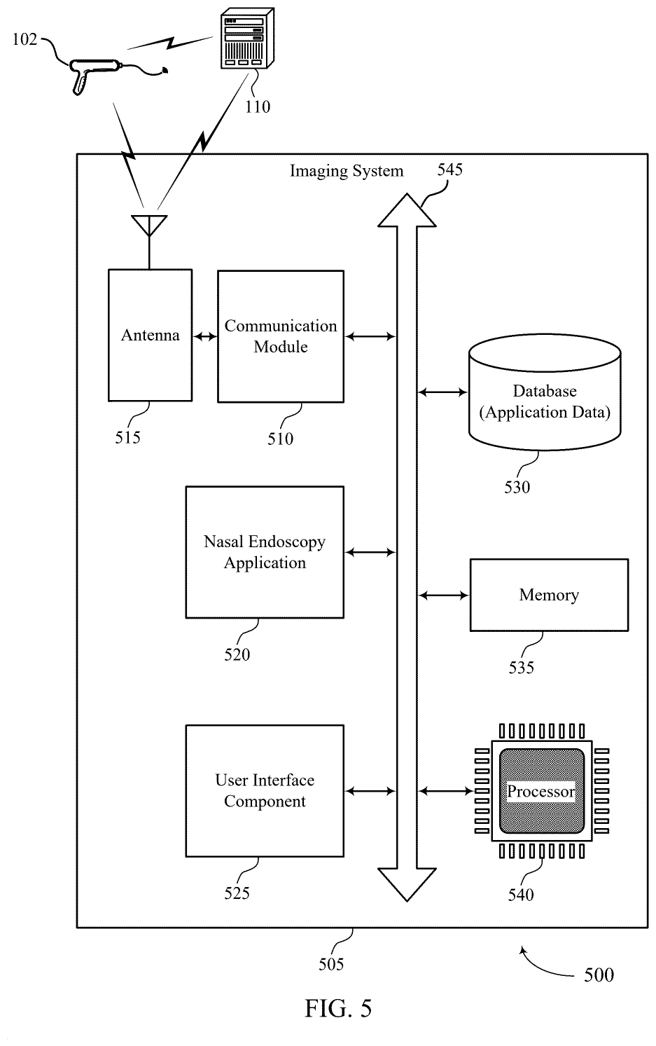

FIG. 5 shows a diagram of a system 500 including a device 505 that supports techniques for nasal endoscopy procedure guidance and diagnostics in accordance with aspects of the present disclosure. The device 505 may be an example of or include components of a device 305 as described herein. The device 505 may include components for bi-directional communications including components for transmitting and receiving communications with a nasal endoscopy device 102, a server, and/or processors 110, such as a nasal endoscopy application 520, a communication module 510, one or more antennas 515, a user interface component 525, a database (application data) 530, at least one memory 535, and at least one processor 540. These components may be in electronic communication or otherwise coupled (e.g., operatively, communicatively, functionally, electronically, electrically) via one or more buses (e.g., a bus 545).

The communication module 510 may manage input and output signals for the device 505 via the antenna 515. The communication module 510 may also manage peripherals not integrated into the device 505. In some cases, the communication module 510 may represent a physical connection or port to an external peripheral. In some cases, the communication module 510 may utilize an operating system such as IOS®, ANDROID®, MS-DOS®, MS-WINDOWS®, OS/2®, UNIX®, LINUX®, or another known operating system. In some cases, the communication module 510 may be implemented as part of the processor 540. In some examples, a user may interact with the device 505 via the communication module 510, user interface component 525, or via hardware components controlled by the communication module 510.

In some cases, the device 505 may include a single antenna 515. However, in some other cases, the device 505 may have more than one antenna 515, which may be capable of concurrently transmitting or receiving multiple wireless transmissions. The communication module 510 may communicate bi-directionally, via the one or more antennas 515, wired, or wireless links as described herein. For example, the communication module 510 may represent a wireless transceiver and may communicate bi-directionally with another wireless transceiver. The communication module 510 may also include a modem to modulate the packets, to provide the modulated packets to one or more antennas 515 for transmission, and to demodulate packets received from the one or more antennas 515.

The user interface component 525 may manage data storage and processing in a database 530. In some cases, a user may interact with the user interface component 525. In other cases, the user interface component 525 may operate automatically without user interaction. The database 530 may be an example of a single database, a distributed database, multiple distributed databases, a data store, a data lake, or an emergency backup database.

The memory 535 may include RAM and ROM. The memory 535 may store computer-readable, computer-executable software including instructions that, when executed, cause the processor 540 to perform various functions described herein. In some cases, the memory 535 may contain, among other things, a BIOS which may control basic hardware or software operation such as the interaction with peripheral components or devices.

The processor 540 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 540 may be configured to operate a memory array using a memory controller. In other cases, a memory controller may be integrated into the processor 540. The processor 540 may be configured to execute computer-readable instructions stored in a memory 535 to perform various functions (e.g., functions or tasks supporting a method and system for sleep staging algorithms).

The nasal endoscopy application 520 may support providing feedback associated with a nasal endoscopy procedure in accordance with examples as disclosed herein. For example, the nasal endoscopy application 520 may be configured as or otherwise support a means for acquiring a plurality of images of a nasal cavity of a user using a nasal endoscopy device. The nasal endoscopy application 520 may be configured as or otherwise support a means for transmitting, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both. The nasal endoscopy application 520 may be configured as or otherwise support a means for inputting, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users. The nasal endoscopy application 520 may be configured as or otherwise support a means for determining, using the one or more machine learning models, an anatomical location of the nasal endoscopy device within the nasal cavity of the user based at least in part on inputting the plurality of images into the one or more machine learning models. The nasal endoscopy application 520 may be configured as or otherwise support a means for transmitting, to the GUI of the imaging system and based at least in part on the determined anatomical location, a second instruction configured to cause the GUI to display endoscope feedback associated with the plurality of images, wherein the endoscope feedback comprises an indication of the anatomical location of the nasal endoscopy device, an instruction for moving the nasal endoscopy device relative to the anatomical location, an indication of an anatomical structure depicted within the plurality of images, a diagnosis of a medical condition within the nasal cavity depicted within the plurality of images, or any combination thereof.

Additionally, or alternatively, the nasal endoscopy application 520 may support providing feedback associated with a nasal endoscopy procedure in accordance with examples as disclosed herein. For example, the nasal endoscopy application 520 may be configured as or otherwise support a means for acquiring a plurality of images of a nasal cavity of a user using a nasal endoscopy device. The nasal endoscopy application 520 may be configured as or otherwise support a means for transmitting, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both. The nasal endoscopy application 520 may be configured as or otherwise support a means for inputting, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users. The nasal endoscopy application 520 may be configured as or otherwise support a means for generating endoscope feedback associated with the plurality of images based at least in part on inputting the plurality of images into the one or more machine learning models. The nasal endoscopy application 520 may be configured as or otherwise support a means for transmitting, to the GUI of the imaging system, a second instruction configured to cause the GUI to display the endoscope feedback associated with the plurality of images, wherein the endoscope feedback comprises an indication of an anatomical location of the nasal endoscopy device within the nasal cavity, an instruction for moving the nasal endoscopy device relative to the anatomical location, an indication of an anatomical structure depicted within the plurality of images, a diagnosis of a medical condition within the nasal cavity depicted within the plurality of images, or any combination thereof.

The nasal endoscopy application 520 may include an application (e.g., “app”), program, software, or other component which is configured to facilitate communications with an imaging system 106, a GUI 108, processors 110, a nasal endoscopy device 102, and the like.

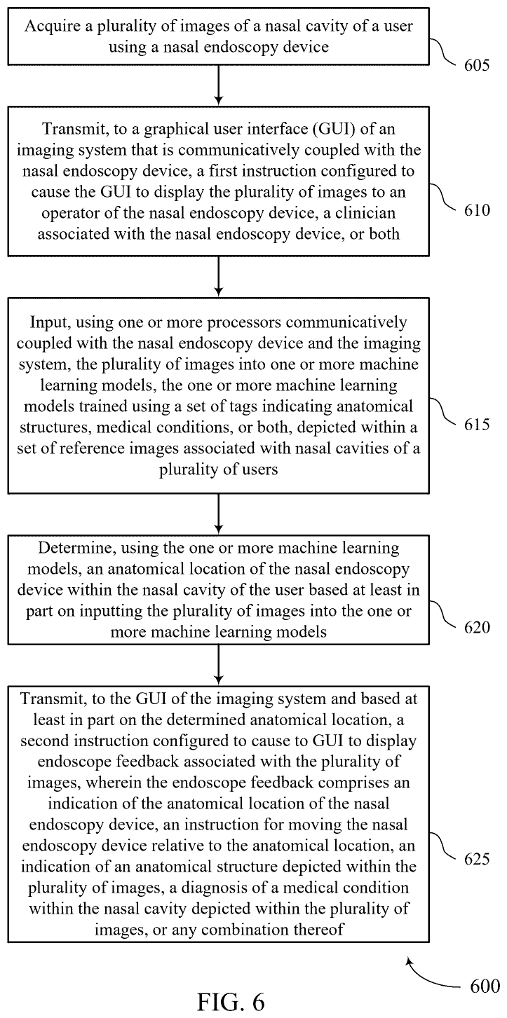

FIG. 6 shows a flowchart illustrating a method 600 that supports techniques for nasal endoscopy procedure guidance and diagnostics in accordance with aspects of the present disclosure. The operations of the method 600 may be performed by a system as described with reference to FIGS. 1 through 5. In some examples, a system may execute a set of instructions to control the functional elements of the user device to perform the described functions. Additionally, or alternatively, the user device may perform aspects of the described functions using special-purpose hardware.

At 605, the method may include acquiring a plurality of images of a nasal cavity of a user using a nasal endoscopy device. The operations of 605 may be performed in accordance with examples as disclosed herein. In some examples, aspects of the operations of 605 may be performed by a nasal endoscopy device manager 425 as described with reference to FIG. 4.

At 610, the method may include transmitting, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both. The operations of 610 may be performed in accordance with examples as disclosed herein. In some examples, aspects of the operations of 610 may be performed by a GUI communication manager 430 as described with reference to FIG. 4.

At 615, the method may include inputting, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users. The operations of 615 may be performed in accordance with examples as disclosed herein. In some examples, aspects of the operations of 615 may be performed by a machine learning model manager 435 as described with reference to FIG. 4.

At 620, the method may include determining, using the one or more machine learning models, an anatomical location of the nasal endoscopy device within the nasal cavity of the user based at least in part on inputting the plurality of images into the one or more machine learning models. The operations of 620 may be performed in accordance with examples as disclosed herein. In some examples, aspects of the operations of 620 may be performed by a machine learning model manager 435 as described with reference to FIG. 4.

At 625, the method may include transmitting, to the GUI of the imaging system and based at least in part on the determined anatomical location, a second instruction configured to cause the GUI to display endoscope feedback associated with the plurality of images, wherein the endoscope feedback comprises an indication of the anatomical location of the nasal endoscopy device, an instruction for moving the nasal endoscopy device relative to the anatomical location, an indication of an anatomical structure depicted within the plurality of images, a clinical guidance or diagnosis of a medical condition within the nasal cavity depicted within the plurality of images, or any combination thereof. The operations of 625 may be performed in accordance with examples as disclosed herein. In some examples, aspects of the operations of 625 may be performed by an endoscope feedback manager 440 as described with reference to FIG. 4.

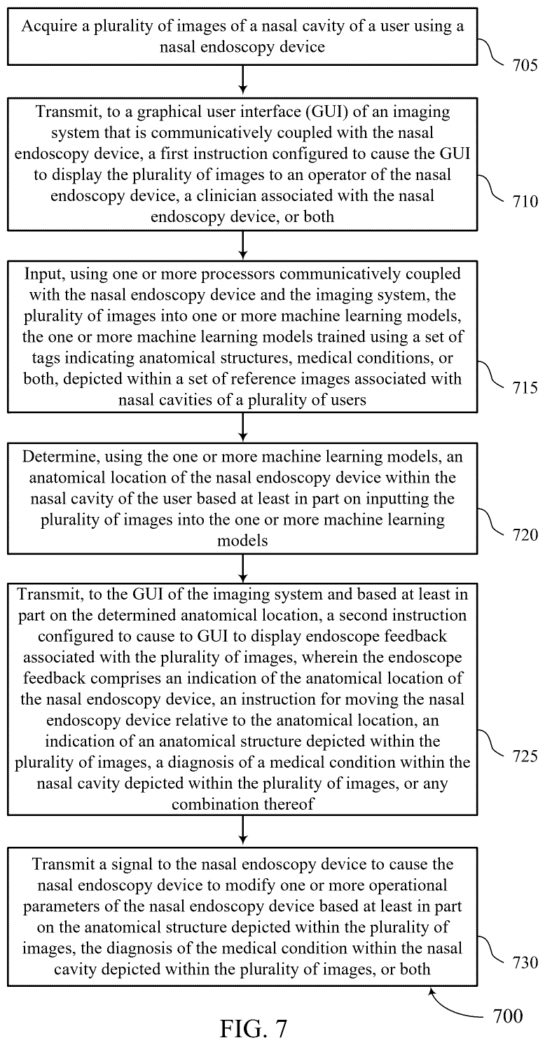

FIG. 7 shows a flowchart illustrating a method 700 that supports techniques for nasal endoscopy procedure guidance and diagnostics in accordance with aspects of the present disclosure. The operations of the method 700 may be performed by a system as described with reference to FIGS. 1 through 5. In some examples, a system may execute a set of instructions to control the functional elements of the user device to perform the described functions. Additionally, or alternatively, the user device may perform aspects of the described functions using special-purpose hardware.

At 705, the method may include acquiring a plurality of images of a nasal cavity of a user using a nasal endoscopy device. The operations of 705 may be performed in accordance with examples as disclosed herein. In some examples, aspects of the operations of 705 may be performed by a nasal endoscopy device manager 425 as described with reference to FIG. 4.

At 710, the method may include transmitting, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both. The operations of 710 may be performed in accordance with examples as disclosed herein. In some examples, aspects of the operations of 710 may be performed by a GUI communication manager 430 as described with reference to FIG. 4.

At 715, the method may include inputting, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users. The operations of 715 may be performed in accordance with examples as disclosed herein. In some examples, aspects of the operations of 715 may be performed by a machine learning model manager 435 as described with reference to FIG. 4.

At 720, the method may include determining, using the one or more machine learning models, an anatomical location of the nasal endoscopy device within the nasal cavity of the user based at least in part on inputting the plurality of images into the one or more machine learning models. The operations of 720 may be performed in accordance with examples as disclosed herein. In some examples, aspects of the operations of 720 may be performed by a machine learning model manager 435 as described with reference to FIG. 4.

At 725, the method may include transmitting, to the GUI of the imaging system and based at least in part on the determined anatomical location, a second instruction configured to cause the GUI to display endoscope feedback associated with the plurality of images, wherein the endoscope feedback comprises an indication of the anatomical location of the nasal endoscopy device, an instruction for moving the nasal endoscopy device relative to the anatomical location, an indication of an anatomical structure depicted within the plurality of images, a clinical guidance or diagnosis of a medical condition within the nasal cavity depicted within the plurality of images, or any combination thereof. The operations of 725 may be performed in accordance with examples as disclosed herein. In some examples, aspects of the operations of 725 may be performed by an endoscope feedback manager 440 as described with reference to FIG. 4.

At 730, the method may include transmitting a signal to the nasal endoscopy device to cause the nasal endoscopy device to modify one or more operational parameters of the nasal endoscopy device based at least in part on the anatomical structure depicted within the plurality of images, the diagnosis of the medical condition within the nasal cavity depicted within the plurality of images, or both. The operations of 730 may be performed in accordance with examples as disclosed herein. In some examples, aspects of the operations of 730 may be performed by a nasal endoscopy device manager 425 as described with reference to FIG. 4.

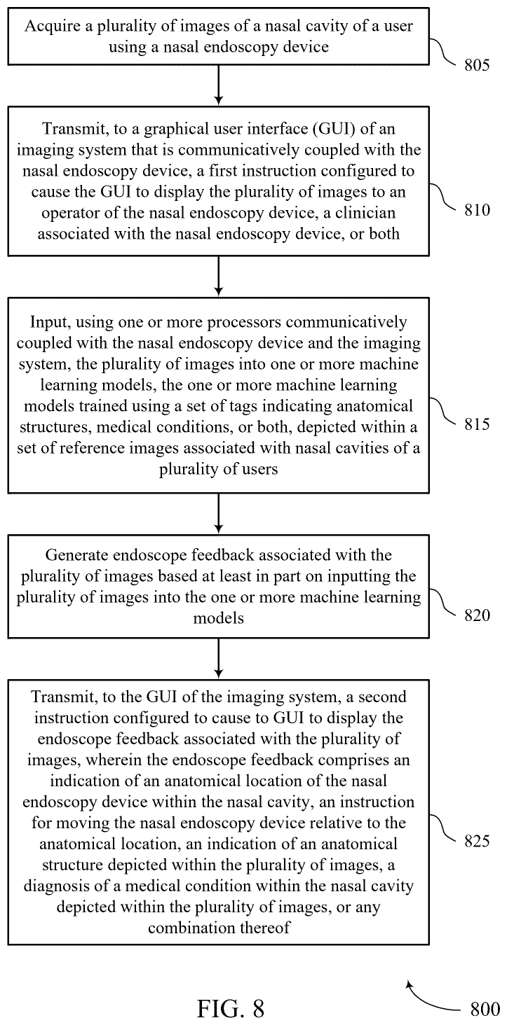

FIG. 8 shows a flowchart illustrating a method 800 that supports techniques for nasal endoscopy procedure guidance and diagnostics in accordance with aspects of the present disclosure. The operations of the method 800 may be implemented by a system or its components as described herein. In some examples, a system may execute a set of instructions to control the functional elements of the user device to perform the described functions. Additionally, or alternatively, the user device may perform aspects of the described functions using special-purpose hardware.

At 805, the method may include acquiring a plurality of images of a nasal cavity of a user using a nasal endoscopy device. The operations of 805 may be performed in accordance with examples as disclosed herein. In some examples, aspects of the operations of 805 may be performed by a nasal endoscopy device manager 425 as described with reference to FIG. 4.

At 810, the method may include transmitting, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both. The operations of 810 may be performed in accordance with examples as disclosed herein. In some examples, aspects of the operations of 810 may be performed by a GUI communication manager 430 as described with reference to FIG. 4.

At 815, the method may include inputting, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users. The operations of 815 may be performed in accordance with examples as disclosed herein. In some examples, aspects of the operations of 815 may be performed by a machine learning model manager 435 as described with reference to FIG. 4.

At 820, the method may include generating endoscope feedback associated with the plurality of images based at least in part on inputting the plurality of images into the one or more machine learning models. The operations of 820 may be performed in accordance with examples as disclosed herein. In some examples, aspects of the operations of 820 may be performed by an endoscope feedback manager 440 as described with reference to FIG. 4.

At 825, the method may include transmitting, to the GUI of the imaging system, a second instruction configured to cause the GUI to display the endoscope feedback associated with the plurality of images, wherein the endoscope feedback comprises an indication of an anatomical location of the nasal endoscopy device within the nasal cavity, an instruction for moving the nasal endoscopy device relative to the anatomical location, an indication of an anatomical structure depicted within the plurality of images, a clinical guidance or diagnosis of a medical condition within the nasal cavity depicted within the plurality of images, or any combination thereof. The operations of 825 may be performed in accordance with examples as disclosed herein. In some examples, aspects of the operations of 825 may be performed by an endoscope feedback manager 440 as described with reference to FIG. 4.

It should be noted that the methods described above describe possible implementations, and that the operations and the steps may be rearranged or otherwise modified and that other implementations are possible. Furthermore, aspects from two or more of the methods may be combined.

A method for providing feedback associated with a nasal endoscopy procedure by an apparatus is described. The method may include acquiring a plurality of images of a nasal cavity of a user using a nasal endoscopy device, transmitting, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both, inputting, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users, determining, using the one or more machine learning models, an anatomical location of the nasal endoscopy device within the nasal cavity of the user based at least in part on inputting the plurality of images into the one or more machine learning models, and transmitting, to the GUI of the imaging system and based at least in part on the determined anatomical location, a second instruction configured to cause the GUI to display endoscope feedback associated with the plurality of images, wherein the endoscope feedback comprises an indication of the anatomical location of the nasal endoscopy device, an instruction for moving the nasal endoscopy device relative to the anatomical location, an indication of an anatomical structure depicted within the plurality of images, a diagnosis of a medical condition within the nasal cavity depicted within the plurality of images, or any combination thereof.

An apparatus for providing feedback associated with a nasal endoscopy procedure is described. The apparatus may include one or more memories storing processor executable code, and one or more processors coupled with the one or more memories. The one or more processors may individually or collectively be operable to execute the code to cause the apparatus to acquire a plurality of images of a nasal cavity of a user using a nasal endoscopy device, transmit, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both, input, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users, determine, using the one or more machine learning models, an anatomical location of the nasal endoscopy device within the nasal cavity of the user based at least in part on inputting the plurality of images into the one or more machine learning models, and transmit, to the GUI of the imaging system and based at least in part on the determined anatomical location, a second instruction configured to cause the GUI to display endoscope feedback associated with the plurality of images, wherein the endoscope feedback comprises an indication of the anatomical location of the nasal endoscopy device, an instruction for moving the nasal endoscopy device relative to the anatomical location, an indication of an anatomical structure depicted within the plurality of images, a diagnosis of a medical condition within the nasal cavity depicted within the plurality of images, or any combination thereof.

Another apparatus for providing feedback associated with a nasal endoscopy procedure is described. The apparatus may include means for acquiring a plurality of images of a nasal cavity of a user using a nasal endoscopy device, means for transmitting, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both, means for inputting, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users, means for determining, using the one or more machine learning models, an anatomical location of the nasal endoscopy device within the nasal cavity of the user based at least in part on inputting the plurality of images into the one or more machine learning models, and means for transmitting, to the GUI of the imaging system and based at least in part on the determined anatomical location, a second instruction configured to cause the GUI to display endoscope feedback associated with the plurality of images, wherein the endoscope feedback comprises an indication of the anatomical location of the nasal endoscopy device, an instruction for moving the nasal endoscopy device relative to the anatomical location, an indication of an anatomical structure depicted within the plurality of images, a diagnosis of a medical condition within the nasal cavity depicted within the plurality of images, or any combination thereof.

A non-transitory computer-readable medium storing code for providing feedback associated with a nasal endoscopy procedure is described. The code may include instructions executable by one or more processors to acquire a plurality of images of a nasal cavity of a user using a nasal endoscopy device, transmit, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both, input, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users, determine, using the one or more machine learning models, an anatomical location of the nasal endoscopy device within the nasal cavity of the user based at least in part on inputting the plurality of images into the one or more machine learning models, and transmit, to the GUI of the imaging system and based at least in part on the determined anatomical location, a second instruction configured to cause the GUI to display endoscope feedback associated with the plurality of images, wherein the endoscope feedback comprises an indication of the anatomical location of the nasal endoscopy device, an instruction for moving the nasal endoscopy device relative to the anatomical location, an indication of an anatomical structure depicted within the plurality of images, a diagnosis of a medical condition within the nasal cavity depicted within the plurality of images, or any combination thereof.

Some examples of the method, apparatus, and non-transitory computer-readable medium described herein may further include operations, features, means, or instructions for transmitting a signal to the nasal endoscopy device to cause the nasal endoscopy device to modify one or more operational parameters of the nasal endoscopy device based at least in part on the anatomical structure depicted within the plurality of images, the diagnosis of the medical condition within the nasal cavity depicted within the plurality of images, or both.

Some examples of the method, apparatus, and non-transitory computer-readable medium described herein may further include operations, features, means, or instructions for determining, using the one or more machine learning models, a first set of characteristics associated with the anatomical structure depicted within the plurality of images, acquiring additional images of the anatomical structure collected via the nasal endoscopy device using the one or more modified operational parameters and based at least in part on transmitting the signal to the nasal endoscopy device, inputting the additional images into the one or more machine learning models, and determining, using the one or more machine learning models, a second set of characteristics associated with the anatomical structure depicted within the additional images.

Some examples of the method, apparatus, and non-transitory computer-readable medium described herein may further include operations, features, means, or instructions for acquiring additional images of the nasal cavity of the user collected via the nasal endoscopy device using the one or more modified operational parameters and based at least in part on transmitting the signal to the nasal endoscopy device, inputting the additional images into the one or more machine learning models, and determining, using the one or more machine learning models, an adjusted diagnosis of the medical condition based at least in part on inputting the additional images into the one or more machine learning models.

Some examples of the method, apparatus, and non-transitory computer-readable medium described herein may further include operations, features, means, or instructions for acquiring additional images of the nasal cavity of the user collected via the nasal endoscopy device using the one or more modified operational parameters and based at least in part on transmitting the signal to the nasal endoscopy device, inputting the additional images into the one or more machine learning models, and transmitting, to the GUI of the imaging system and based at least in part on inputting the additional images into the one or more machine learning models, a third instruction configured to cause the GUI to display additional endoscope feedback associated with the additional images.

In some examples of the method, apparatus, and non-transitory computer-readable medium described herein, the one or more operational parameters of the nasal endoscopy device comprise a wavelength of light used by one or more light-emitting components of the nasal endoscopy device, a brightness of the one or more light-emitting components, a frame rate for collecting images, a color saturation setting, one or more imaging settings of one or more light-receiving components of the nasal endoscopy device, or any combination thereof.

Some examples of the method, apparatus, and non-transitory computer-readable medium described herein may further include operations, features, means, or instructions for identifying, using the one or more machine learning models, an image of the plurality of images that depicts a reference structure within the nasal cavity of the user and generating an association between the image and a reference anatomical location, wherein determining the anatomical location of the nasal endoscopy device within the nasal cavity may be based at least in part on the reference anatomical location and a comparison between the image and the plurality of images.

In some examples of the method, apparatus, and non-transitory computer-readable medium described herein, the reference structure comprises a middle meatus of the user.

Some examples of the method, apparatus, and non-transitory computer-readable medium described herein may further include operations, features, means, or instructions for receiving speed data, acceleration data, or both, associated with a movement of the nasal endoscopy device within the nasal cavity of the user and inputting the speed data, the acceleration data, or both, into the one or more machine learning models, wherein the anatomical location of the nasal endoscopy device may be determined based at least in part on the reference anatomical location and the speed data, the acceleration data, or both.

Some examples of the method, apparatus, and non-transitory computer-readable medium described herein may further include operations, features, means, or instructions for receiving speed data, acceleration data, or both, associated with a movement of the nasal endoscopy device within the nasal cavity during the nasal endoscopy procedure, inputting the speed data, the acceleration data, or both, into the one or more machine learning models, and generating, using the one or more machine learning models, a three-dimensional model of the nasal cavity of the user based at least in part on the plurality of images and the speed data, the acceleration data, or both, wherein determining the anatomical location of the nasal endoscopy device may be based at least in part on the three-dimensional model.

Some examples of the method, apparatus, and non-transitory computer-readable medium described herein may further include operations, features, means, or instructions for identifying a plurality of anatomical structures or anatomical locations of the nasal cavity of the user depicted within the plurality of images and comparing the plurality of anatomical structures or anatomical locations of the nasal cavity of the user with a plurality of reference structures or reference locations, wherein the endoscope feedback may be based at least in part on the comparison.

In some examples of the method, apparatus, and non-transitory computer-readable medium described herein, the endoscope feedback comprises an indication of a completion of the nasal endoscopy procedure based at least in part on the plurality of anatomical structures or anatomical locations matching the plurality of reference structures or reference locations and the endoscope feedback comprises directions for the operator of the nasal endoscopy device move the nasal endoscopy device to a different anatomical location based at least in part on the plurality of anatomical structures or anatomical locations failing to match the plurality of reference structures or reference locations.

Some examples of the method, apparatus, and non-transitory computer-readable medium described herein may further include operations, features, means, or instructions for determining, using the one or more machine learning models, a difference between a viewpoint of the anatomical structure of the user depicted within the plurality of images and a reference viewpoint of one or more corresponding anatomical structures of the plurality of users depicted within the set of reference images, wherein the endoscope feedback comprises directions for the operator to manipulate the nasal endoscopy device to match the viewpoint of the anatomical structure with the reference viewpoint.

In some examples of the method, apparatus, and non-transitory computer-readable medium described herein, the endoscope feedback comprises directions for the operator to maintain the nasal endoscopy device in a current anatomical location based at least in part on the anatomical structure depicted within the plurality of images.

Some examples of the method, apparatus, and non-transitory computer-readable medium described herein may further include operations, features, means, or instructions for determining, using the one or more machine learning models, a relative priority of the plurality of images based at least in part on the anatomical location associated with each of the plurality of images, anatomical structures depicted within each of the plurality of images, or both.

Some examples of the method, apparatus, and non-transitory computer-readable medium described herein may further include operations, features, means, or instructions for acquiring, during a reference nasal endoscopy procedure, a plurality of reference images of the nasal cavity of the user and determining, using the one or more machine learning models and based on a comparison between the plurality of images and the plurality of reference images, a change in one or more characteristics of the anatomical structure, a change in the diagnosis of the medical condition, or both.

In some examples of the method, apparatus, and non-transitory computer-readable medium described herein, the second instruction causes the GUI of the imaging system to overlay the endoscope feedback on top of the plurality of images in real time or near-real time during the nasal endoscopy procedure.

In some examples of the method, apparatus, and non-transitory computer-readable medium described herein, the anatomical structure comprises a structural feature of the nasal cavity, a polyp, or both.

Some examples of the method, apparatus, and non-transitory computer-readable medium described herein may further include operations, features, means, or instructions for receiving the set of reference images depicting the nasal cavities of the plurality of users, tagging the set of reference images with the set of tags based on the anatomical structures, medical conditions, or both, depicted in the respective images to generate a set of tagged reference images, and training the one or more machine learning models to diagnose medical conditions during nasal endoscopy procedures based at least in part on inputting the set of tagged reference images into the one or more machine learning models, wherein determining the anatomical location of the plurality of images may be based at least in part on comparing, using the one or more machine learning models, the plurality of images with the set of tagged reference images.

In some examples of the method, apparatus, and non-transitory computer-readable medium described herein, the nasal endoscopy device comprises an otoscope, a nasoendoscope, a rhinoscope, a laryngoscope, or any combination thereof.

Some examples of the method, apparatus, and non-transitory computer-readable medium described herein may further include operations, features, means, or instructions for comparing the plurality of images with the set of reference images using the one or more machine learning models, selecting, based at least in part on the comparison, at least one image from the set of reference images that satisfies one or more similarity criteria with the plurality of images, and transmitting, to the GUI of the imaging system, a third instruction configured to cause the GUI to display the selected at least one image alongside the plurality of images.

A method for providing feedback associated with a nasal endoscopy procedure by an apparatus is described. The method may include acquiring a plurality of images of a nasal cavity of a user using a nasal endoscopy device, transmitting, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both, inputting, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users, generating endoscope feedback associated with the plurality of images based at least in part on inputting the plurality of images into the one or more machine learning models, and transmitting, to the GUI of the imaging system, a second instruction configured to cause the GUI to display the endoscope feedback associated with the plurality of images, wherein the endoscope feedback comprises an indication of an anatomical location of the nasal endoscopy device within the nasal cavity, an instruction for moving the nasal endoscopy device relative to the anatomical location, an indication of an anatomical structure depicted within the plurality of images, a diagnosis of a medical condition within the nasal cavity depicted within the plurality of images, or any combination thereof.

An apparatus for providing feedback associated with a nasal endoscopy procedure is described. The apparatus may include one or more memories storing processor executable code, and one or more processors coupled with the one or more memories. The one or more processors may individually or collectively be operable to execute the code to cause the apparatus to acquire a plurality of images of a nasal cavity of a user using a nasal endoscopy device, transmit, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both, input, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users, generate endoscope feedback associated with the plurality of images based at least in part on inputting the plurality of images into the one or more machine learning models, and transmit, to the GUI of the imaging system, a second instruction configured to cause the GUI to display the endoscope feedback associated with the plurality of images, wherein the endoscope feedback comprises an indication of an anatomical location of the nasal endoscopy device within the nasal cavity, an instruction for moving the nasal endoscopy device relative to the anatomical location, an indication of an anatomical structure depicted within the plurality of images, a diagnosis of a medical condition within the nasal cavity depicted within the plurality of images, or any combination thereof.

Another apparatus for providing feedback associated with a nasal endoscopy procedure is described. The apparatus may include means for acquiring a plurality of images of a nasal cavity of a user using a nasal endoscopy device, means for transmitting, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both, means for inputting, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users, means for generating endoscope feedback associated with the plurality of images based at least in part on inputting the plurality of images into the one or more machine learning models, and means for transmitting, to the GUI of the imaging system, a second instruction configured to cause the GUI to display the endoscope feedback associated with the plurality of images, wherein the endoscope feedback comprises an indication of an anatomical location of the nasal endoscopy device within the nasal cavity, an instruction for moving the nasal endoscopy device relative to the anatomical location, an indication of an anatomical structure depicted within the plurality of images, a diagnosis of a medical condition within the nasal cavity depicted within the plurality of images, or any combination thereof.

A non-transitory computer-readable medium storing code for providing feedback associated with a nasal endoscopy procedure is described. The code may include instructions executable by one or more processors to acquire a plurality of images of a nasal cavity of a user using a nasal endoscopy device, transmit, to a GUI of an imaging system that is communicatively coupled with the nasal endoscopy device, a first instruction configured to cause the GUI to display the plurality of images to an operator of the nasal endoscopy device, a clinician associated with the nasal endoscopy device, or both, input, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users, generate endoscope feedback associated with the plurality of images based at least in part on inputting the plurality of images into the one or more machine learning models, and transmit, to the GUI of the imaging system, a second instruction configured to cause the GUI to display the endoscope feedback associated with the plurality of images, wherein the endoscope feedback comprises an indication of an anatomical location of the nasal endoscopy device within the nasal cavity, an instruction for moving the nasal endoscopy device relative to the anatomical location, an indication of an anatomical structure depicted within the plurality of images, a diagnosis of a medical condition within the nasal cavity depicted within the plurality of images, or any combination thereof.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A system for providing feedback associated with a nasal endoscopy procedure, comprising:
an imaging system communicatively coupled with a nasal endoscopy device, the imaging system comprising a graphical user interface (GUI); and
one or more processors communicatively coupled with the nasal endoscopy device and the imaging system, the one or more processors configured to:
acquire a plurality of images of a nasal cavity of a user collected via the nasal endoscopy device;
transmit, to the GUI of the imaging system, a first instruction configured to cause the GUI to display the plurality of images to an operator of the nasal endoscopy device, a clinician associated with the nasal endoscopy device, or both;
input the plurality of images into one or more machine learning models, the one or more machine learning models trained using a set of tags indicating anatomical structures, medical conditions, or both, depicted within a set of reference images associated with nasal cavities or other anatomical locations of a plurality of users;
determine, using the one or more machine learning models, an anatomical location of the nasal endoscopy device within the nasal cavity of the user based at least in part on inputting the plurality of images into the one or more machine learning models; and
transmit, to the GUI of the imaging system and based at least in part on the determined anatomical location, a second instruction configured to cause the GUI to display endoscope feedback associated with the plurality of images, wherein the endoscope feedback comprises an indication of the anatomical location of the nasal endoscopy device, an instruction for moving the nasal endoscopy device relative to the anatomical location, an indication of an anatomical structure depicted within the plurality of images, a clinical guidance or diagnosis of a medical condition within the nasal cavity depicted within the plurality of images, or any combination thereof.

2. The system of claim 1, wherein the one or more processors are further configured to:
transmit a signal to the nasal endoscopy device to cause the nasal endoscopy device to modify one or more operational parameters of the nasal endoscopy device based at least in part on the anatomical structure depicted within the plurality of images, the clinical guidance or diagnosis of the medical condition within the nasal cavity depicted within the plurality of images, or both.

3. The system of claim 2, wherein the one or more processors are further configured to:
determine, using the one or more machine learning models, a first set of characteristics associated with the anatomical structure depicted within the plurality of images;
acquire additional images of the anatomical structure collected via the nasal endoscopy device using the one or more modified operational parameters and based at least in part on transmitting the signal to the nasal endoscopy device;
input the additional images information into the one or more machine learning models; and
determine, using the one or more machine learning models, a second set of characteristics associated with the anatomical structure depicted within the additional images.

4. The system of claim 2, wherein the one or more processors are further configured to:
acquire additional images of the nasal cavity of the user collected via the nasal endoscopy device using the one or more modified operational parameters and based at least in part on transmitting the signal to the nasal endoscopy device;
input the additional images into the one or more machine learning models; and
determine, using the one or more machine learning models, an adjusted clinical guidance or adjusted diagnosis of the medical condition based at least in part on inputting the additional images into the one or more machine learning models.

5. The system of claim 2, wherein the one or more processors are further configured to:
acquire additional images of the nasal cavity of the user collected via the nasal endoscopy device using the one or more modified operational parameters and based at least in part on transmitting the signal to the nasal endoscopy device;
input the additional images into the one or more machine learning models; and
transmit, to the GUI of the imaging system and based at least in part on inputting the additional images into the one or more machine learning models, a third instruction configured to cause the GUI to display additional endoscope feedback associated with the additional images.

6. The system of claim 2, wherein the one or more operational parameters of the nasal endoscopy device comprise a wavelength of light used by one or more light-emitting components of the nasal endoscopy device, a brightness of the one or more light-emitting components, a frame rate for collecting images, a color saturation setting, a level of suction, an irrigation setting, a medication delivery setting, one or more imaging settings of one or more light-receiving components of the nasal endoscopy device, or any combination thereof.

7. The system of claim 1, wherein the one or more processors are further configured to:
identify, using the one or more machine learning models, an image of the plurality of images that depicts a reference structure within the nasal cavity of the user; and
generate an association between the image and a reference anatomical location, wherein determining the anatomical location of the nasal endoscopy device within the nasal cavity is based at least in part on the reference anatomical location and a comparison between the image and the plurality of images.

8. The system of claim 7, wherein the reference structure comprises a middle meatus of the user.

9. The system of claim 7, wherein the one or more processors are further configured to:
receive speed data, acceleration data, or both, associated with a movement of the nasal endoscopy device within the nasal cavity of the user; and
input the speed data, the acceleration data, or both, into the one or more machine learning models, wherein the anatomical location of the nasal endoscopy device is determined based at least in part on the reference anatomical location and the speed data, the acceleration data, or both.

10. The system of claim 1, wherein the one or more processors are further configured to:
receive speed data, acceleration data, or both, associated with a movement of the nasal endoscopy device within the nasal cavity during the nasal endoscopy procedure;
input the speed data, the acceleration data, or both, into the one or more machine learning models; and
generate, using the one or more machine learning models, a three-dimensional model of the nasal cavity of the user based at least in part on the plurality of images and the speed data, the acceleration data, or both, wherein determining the anatomical location of the nasal endoscopy device is based at least in part on the three-dimensional model.

11. The system of claim 1, wherein the one or more processors are further configured to:
identify a plurality of anatomical structures or anatomical locations of the nasal cavity of the user depicted within the plurality of images; and
compare the plurality of anatomical structures or anatomical locations of the nasal cavity of the user with a plurality of reference structures or reference locations, wherein the endoscope feedback is based at least in part on the comparison,
wherein the endoscope feedback comprises an indication of a completion of the nasal endoscopy procedure based at least in part on the plurality of anatomical structures or anatomical locations matching the plurality of reference structures or reference locations, or
wherein the endoscope feedback comprises directions for the operator of the nasal endoscopy device move the nasal endoscopy device to a different anatomical location based at least in part on the plurality of anatomical structures or anatomical locations failing to match the plurality of reference structures or reference locations.

12. The system of claim 1, wherein the endoscope feedback is further based at least in part on additional data associated with the user, wherein the additional data comprises a blood oxygen saturation level associated with the user, a respiration rate associated with the user, a blood pressure associated with the user, or any combination thereof.

13. The system of claim 1, wherein the one or more processors are further configured to:
determine, using the one or more machine learning models, a difference between a viewpoint of the anatomical structure of the user depicted within the plurality of images and a reference viewpoint of one or more corresponding anatomical structures of the plurality of users depicted within the set of reference images, wherein the endoscope feedback comprises directions for the operator to manipulate the nasal endoscopy device to match the viewpoint of the anatomical structure with the reference viewpoint.

14. The system of claim 1, wherein the one or more processors are further configured to:
acquire, during a reference nasal endoscopy procedure, a plurality of reference images of the nasal cavity of the user; and
determine, using the one or more machine learning models and based on a comparison between the plurality of images and the plurality of reference images, a change in one or more characteristics of the anatomical structure, a change in the clinical guidance or diagnosis of the medical condition, or both.

15. The system of claim 1, wherein the second instruction causes the GUI of the imaging system to overlay the endoscope feedback on top of the plurality of images in real time or near-real time during the nasal endoscopy procedure.

16. The system of claim 1, wherein the anatomical structure comprises a structural feature of the nasal cavity, a polyp, or both.

17. The system of claim 1, wherein the one or more processors are further configured to:
receive the set of reference images depicting the nasal cavities of the plurality of users;
tag the set of reference images with the set of tags based on the anatomical structures, medical conditions, or both, depicted in the respective images to generate a set of tagged reference images; and
train the one or more machine learning models to identify medical conditions during nasal endoscopy procedures based at least in part on inputting the set of tagged reference images into the one or more machine learning models, wherein determining the anatomical location of the plurality of images is based at least in part on comparing, using the one or more machine learning models, the plurality of images with the set of tagged reference images.

18. The system of claim 1, wherein the nasal endoscopy device comprises an otoscope, a nasoendoscope, a rhinoscope, a laryngoscope, or any combination thereof.

19. A system for providing feedback associated with a nasal endoscopy procedure, comprising:
an imaging system communicatively coupled with a nasal endoscopy device, the imaging system comprising a graphical user interface (GUI); and
one or more processors communicatively coupled with the nasal endoscopy device and the imaging system, the one or more processors configured to:
acquire a plurality of images of a nasal cavity of a user collected via the nasal endoscopy device;
transmit, to the GUI of the imaging system, a first instruction configured to cause the GUI to display the plurality of images to an operator of the nasal endoscopy device;
input the plurality of images into one or more machine learning models, the one or more machine learning models trained using a set of tags indicating anatomical structures, medical conditions, or both, depicted within a set of reference images associated with nasal cavities of a plurality of users;
generate endoscope feedback associated with the plurality of images based at least in part on inputting the plurality of images into the one or more machine learning models; and
transmit, to the GUI of the imaging system, a second instruction configured to cause the GUI to display the endoscope feedback associated with the plurality of images, wherein the endoscope feedback comprises an indication of an anatomical location of the nasal endoscopy device, an instruction for moving the nasal endoscopy device relative to the anatomical location, an indication of an anatomical structure depicted within the plurality of images, a clinical guidance or diagnosis of a medical condition within the nasal cavity depicted within the plurality of images, or any combination thereof.

20. A method for providing feedback associated with a nasal endoscopy procedure, comprising:
acquiring a plurality of images of a nasal cavity of a user using a nasal endoscopy device;
transmitting, to a graphical user interface (GUI) of an imaging system that is communicatively coupled with the nasal endoscopy device, a first instruction configured to cause the GUI to display the plurality of images to an operator of the nasal endoscopy device, a clinician associated with the nasal endoscopy device, or both;
inputting, using one or more processors communicatively coupled with the nasal endoscopy device and the imaging system, the plurality of images into one or more machine learning models, the one or more machine learning models trained using a set of tags indicating anatomical structures, medical conditions, or both, depicted within a set of reference images associated with nasal cavities of a plurality of users;
determining, using the one or more machine learning models, an anatomical location of the nasal endoscopy device within the nasal cavity of the user based at least in part on inputting the plurality of images into the one or more machine learning models; and
transmitting, to the GUI of the imaging system and based at least in part on the determined anatomical location, a second instruction configured to cause the GUI to display endoscope feedback associated with the plurality of images, wherein the endoscope feedback comprises an indication of the anatomical location of the nasal endoscopy device, an instruction for moving the nasal endoscopy device relative to the anatomical location, an indication of an anatomical structure depicted within the plurality of images, a clinical guidance or diagnosis of a medical condition within the nasal cavity depicted within the plurality of images, or any combination thereof.
